(19) United States
(12) Patent Application Publication
(10) Pub. No.: US 2024/0407818 A1
(43) Pub. Date: Dec. 12, 2024

(54) MIXING SYSTEM WITH SIDE AMPULE BREAKER

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 18/680,669

(22) Filed: May 31, 2024

(30) Foreign Application Priority Data
Jun. 6, 2023 (EP) 23177510.7

(51) Int. Cl. A61B 17/88

(52) U.S. Cl. CPC A61B 17/8836; A61B 2017/8838

(57) ABSTRACT

A device for producing a bone cement, comprising an ampule breaker having a first housing, wherein the first housing comprises a first interior, a holder arranged in the first housing for receiving a glass ampule, a breaking element arranged in the first housing, which is designed and configured to break open a glass ampule which can be received in the holder, a collection region arranged in the first housing, which is designed and configured to receive a liquid from a broken-open glass ampule, a cartridge which is configured to receive a powder component of a bone cement, wherein the cartridge comprises a second housing having a second interior, and a fluid-conducting channel for dispensing a liquid from the collection region into the second interior, wherein the fluid-conducting channel is arranged and configured to retain the liquid in the collection region and to dispense the liquid to the second interior.

### CROSS-REFERENCE TO RELATED APPLICATIONS

This application claims priority pursuant to 35 U.S.C. 119(a) to European Application No. 23177510.7, filed Jun. 6, 2023, which application is incorporated herein by reference in its entirety.

### FIELD OF THE INVENTION

The present invention relates to the field of medical technology, in particular to devices for the preparation of bone cement. In particular, devices for opening glass ampules are described which are suitable for dispensing a liquid from such glass ampules. For example, a monomer liquid can be dispensed to a powder component for producing a polymethylmethacrylate bone cement by means of the devices described herein. Moreover, the devices described herein are generally used for storing and opening glass ampules and for dispensing a liquid contained therein.

### TECHNICAL BACKGROUND

Polymethylmethacrylate (PMMA) bone cements are based on the fundamental work of Charnley. PMMA bone cements frequently comprise a liquid monomer component and a powder component. The monomer component frequently comprises the monomer methyl methacrylate and an activator (N,N-dimethyl-p-toluidine) dissolved therein. The powder component, also referred to as bone cement powder, usually contains one or more polymers produced on the basis of methyl methacrylate and comonomers, such as styrene, methyl acrylate or similar monomers, by polymerization, preferably suspension polymerization, a radiopaque agent and the initiator dibenzoyl peroxide. When the powder component is mixed with the monomer component, a plastically deformable dough, the actual bone cement, is produced as a result of the swelling of the polymers of the powder component in the methyl methacrylate. When the powder component is mixed with the monomer component, the activator N,N-dimethyl-p-toluidine reacts with dibenzoyl peroxide to form radicals. The radicals formed initiate the radical polymerization of the methyl methacrylate. As the polymerization of the methyl methacrylate progresses, the viscosity of the cement dough increases until the cement dough solidifies.

Polymethyl methacrylate bone cements can be mixed in suitable mixing beakers with the aid of spatulas by mixing the cement powder with the monomer liquid. Air bubbles may be incorporated in the bone cement dough, which can negatively influence the mechanical properties of the hardened bone cement. In order to avoid air inclusions in the bone cement dough, a plurality of vacuum cementing systems have been described, of which the following are mentioned by way of example: U.S. Pat. Nos. 6,033,105A, 5,624,184A, 4,671,263A, 4,973,168A, 5,100,241A, WO99/67015A1, EP1 016 452A2, DE36 40 279A1, WO94/26403A1, EP1 005 901A2, and U.S. Pat. No. 5,344,232A.

A further development in cementing technology is represented by cementing systems in which both the cement powder and the monomer liquid are already packaged in separate compartments of the mixing systems and are first mixed with one another in the cementing system immediately before application of the cement. Such closed full-prepacked mixing systems were described in the patents EP0380867B1, EP0796653B1, EP0692229B1, DE102009031178B1, U.S. Pat. Nos. 5,997,544B1, 6,709,149B1, DE69812726B1, and U.S. Pat. No. 5,588,745B1, WO9416951A1, DE19718648A1, EP1741413B1, EP3054880B1, DE102009031178B3, U.S. Pat. No. 8,662,736B2, and EP3093067B1.

EP 1741413B1 describes a mixing system consisting of two cartridges screwed together. Cement powder is located in the interior of the cartridges. The lower cartridge contains a hollow mandrel or two hollow mandrels which are connected to the interior of the second cartridge so as to be permeable to liquid. The tips of the hollow mandrels are closed with a pierceable rubber cap. These must not fall off during storage and transport because otherwise the bone cement powder can exit from the cartridges through the hollow mandrels. One or two bags filled with monomer liquid are located on a rail system on the second cartridge, wherein the bags can be pushed against the hollow mandrels and the hollow mandrels first perforate the rubber caps and then the bags. By applying a vacuum to the two cartridges, the monomer liquid is sucked from the bags into the interior of the two cartridges. The two bone cement components are then mixed with a mixer and then the dispensing plunger is released in the lower cartridge and the latter collects the formed bone cement dough in the upper cartridge, wherein the dispensing plunger moves out of the lower cartridge into the upper cartridge. The lower cartridge is unscrewed from the upper cartridge together with the emptied bags. The upper cartridge is then ready for the bone cement dough to be applied. Monomer liquid bags are in principle suitable containers for storing monomer liquid. However, they are linked to a number of problems. The monomer liquid bags are very complex to produce and to fill aseptically. Furthermore, it is difficult to seal the perforated bags in the region of the hollow mandrels during the monomer fluid transfer. The complete transfer of the monomer liquid from the bags is not always successful because the bags collapse due to the vacuum effect. In this case, residues of the monomer liquid can remain in the collapsed bags.

The monomer liquid of PMMA bone cement can be stored, for example, in glass ampules, and in plastics composite bags, as well as in metallized plastics composite bags.

In order to remove the monomer liquid from a glass ampule, the ampule head can typically be detached from the ampule body and removed. Devices for opening ampules are disclosed in documents DE19532015A1, WO9718031A1, EP1031333A1 and WO2010012114A1.

EP2404864B1 discloses a device in which an ampule is arranged in an ampule holder and wherein the ampule holder is elastically deformable in the region of the ampule neck. The ampule holder is not deformable in the region of the ampule head and the ampule body. By bending the ampule holder over the elastically deformable portion of the ampule holder, which has a hinge function, the ampule head can be pressed against the non-deformable wall of the ampule holder. The ampule head can thereby break off and fall onto a screen. The monomer liquid can then flow out of the opened ampule through the screen. For the device described there to function, it is advantageous that it can be connected in a mechanically stable manner to a mixing system which is to be supplied with monomer liquid, because a considerable tensile load can occur at the attachment to the mixing system during the bending process.

### Preferred Embodiments

The object of the present invention is to solve one or more of the problems described above and further problems of the prior art. For example, the invention facilitates a simple and reliable dispensing of a liquid from a glass ampule to a cartridge having a powder component. Furthermore, the present invention provides a method for dispensing a liquid from a glass ampule to a cartridge having a powder component.

These objects are achieved by the methods and devices described herein, in particular those which are described in the claims.

Preferred embodiments of the invention are described below.

A first aspect relates to a device for producing bone cement, comprising:
an ampule breaker having a first housing, wherein the first housing comprises a first interior,
a holder arranged in the first housing for receiving a glass ampule,
a breaking element which is arranged in the first housing and is designed and configured to break open a glass ampule which can be received in the holder,
a collection region which is arranged in the first housing and is designed and configured to receive a liquid from a broken-open glass ampule,
a cartridge configured to receive a powder component of a bone cement, wherein the cartridge comprises a second housing having a second interior,
a fluid-conducting channel for dispensing a liquid from the collection region into the second interior, wherein the fluid-conducting channel is arranged and configured to retain the liquid in the collection region, and to dispense the liquid to the second interior as soon as a pressure difference is applied between the collection region and the second interior.

A second embodiment describes a device according to embodiment 1, further comprising a catchment region which is arranged in the first housing and configured to receive a portion of a glass ampule that is broken off by the action of the breaking element.

A third embodiment relates to a device according to embodiment 2, further comprising a screen, which is arranged adjacent to the catchment region and is configured to retain fragments from a glass ampule broken open in the device in the ampule breaker.

A fourth embodiment relates to a device according to any of the preceding embodiments, wherein the fluid-conducting channel is arranged at an angle of 60° to 90° relative to the second housing.

A fifth embodiment relates to a device according to any of the preceding embodiments, wherein the fluid-conducting channel has an inner diameter of less than 5 mm, preferably less than 4 mm or more preferably less than 3 mm, at least in portions.

A sixth embodiment relates to a device according to any of the preceding embodiments, wherein a glass ampule is fastened or fixable in a force-fitting, form-fitting or integrally bonded manner in the holder, preferably is fastened or fixable by adhesive bonding or clamping.

A seventh embodiment relates to a device according to embodiment 6, wherein the glass ampule comprises an ampule neck having an annular predetermined breaking point, wherein the inner diameter of the predetermined breaking point is preferably 4 to 7 mm.

An eighth embodiment relates to a device according to any of the preceding embodiments, wherein the cartridge further contains a powder component for preparing a bone cement, wherein the powder component is preferably arranged in the second interior of the second housing.

A ninth embodiment relates to a device according to any of the preceding embodiments, wherein the ampule breaker is designed and configured for simultaneously breaking open and dispensing liquid from two glass ampules to the cartridge.

A tenth embodiment relates to a device according to any of the preceding embodiments, wherein the breaking element comprises a plunger arranged movably in the first housing and a projection, wherein the plunger is designed and configured to break open a glass ampule with a liquid, which glass ampule can be received in the holder, by pressure of the glass ampule against the projection, and to pump the liquid from the glass ampule through the channel out of the first housing into the second housing.

An eleventh embodiment relates to a device according to embodiment 10, wherein the ampule breaker further comprises a valve which is configured to allow air to flow into the first housing from the outside in order to subsequently pump a liquid from a glass ampule, which is broken open in the device, through the channel out of the first housing into the second housing by pressure of the plunger onto the air flowing into the first housing.

A twelfth embodiment relates to a device according to embodiment 11, wherein the valve is a one-way valve, preferably a ball valve, lip valve, or plate valve.

A thirteenth embodiment relates to a device according to any of the preceding embodiments 10 to 12, wherein the ampule breaker is designed and configured to pump the entire contents of a glass ampule received in the holder through the channel out of the first housing into the second housing with a single stroke of the plunger.

A fourteenth embodiment relates to a device according to any of embodiments 1 to 9, wherein the breaking element comprises a button which can be pushed into the first housing from the outside in order to break open a glass ampule, which can be received in the holder, by pushing the button into the first housing, wherein the button is preferably configured to automatically return to its original position after the button has been pushed in.

A further aspect relates to a device according to any of the embodiments 1 to 9, wherein the first housing of the ampule breaker is at least partially flexible in order to break open a glass ampule, which can be received in the holder, by bending the first housing.

### DETAILED DESCRIPTION

With respect to the embodiments described herein, the elements of which “have,” or “comprise,” a particular feature (for example, a material), in principle, a further embodiment is always contemplated in which the relevant element consists solely of the feature, i.e., does not comprise any other constituents. The word “comprise” or “comprising” is used herein synonymously with the word “have” or “having.”

In one embodiment, if an element is denoted by the singular, an embodiment is also contemplated in which more than one such element is present. The use of a term for an element in the plural in principle also encompasses an embodiment in which only a single corresponding element is included.

Unless otherwise indicated or clearly excluded from the context, it is possible in principle, and is hereby clearly contemplated, that features of different embodiments may also be present in the other embodiments described herein. Likewise, it is contemplated in principle that all features described herein in connection with a method are also applicable to the products and devices described herein, and vice versa. All such considered combinations are not explicitly listed in all instances, simply in order to keep the description brief. Technical solutions known to be equivalent to the features described herein are also intended in principle to be encompassed by the scope of the invention.

A first aspect relates to a device for producing bone cement, comprising:
an ampule breaker having a first housing, wherein the first housing comprises a first interior,
a holder arranged in the first housing for receiving a glass ampule,
a breaking element which is arranged in the first housing and is designed and configured to break open a glass ampule which can be received in the holder,
a collection region which is arranged in the first housing and is designed and configured to receive a liquid from a broken-open glass ampule,

a cartridge configured to receive a powder component of a bone cement, wherein the cartridge comprises a second housing having a second interior,
a fluid-conducting channel for dispensing a liquid from the collection region into the second interior, wherein the fluid-conducting channel is arranged and configured to retain the liquid in the collection region, and to dispense the liquid to the second interior as soon as a pressure difference is applied between the collection region and the second interior.

The device comprises an ampule breaker. The ampule breaker preferably forms one of at least two units within the device according to the invention. The ampule breaker is configured to receive a glass ampule with a liquid. The ampule breaker comprises a first housing comprising a first interior. A holder for receiving a glass ampule is arranged within the first housing. Furthermore, the ampule breaker comprises a breaking element which is designed and configured to break open a glass ampule which can be received in the holder. As is shown in more detail below, the breaking element can be designed in different ways. For example, the breaking element can have a plunger and a projection, wherein a glass ampule which can be received in the holder can be pressed against the projection by means of the plunger and can thereby be broken open. The breaking element can also have a button which can be pressed into the first housing from the outside in order thereby to break open a glass ampule which can be received in the holder. The breaking element can also be designed as a flexible part of the housing, wherein by bending the housing, a glass ampule which can be received therein can be broken open. The breaking element can also have other mechanisms known in the art for breaking open a glass ampule. Optionally, a plurality of breaking elements can be provided in the device, for example two pairs of plungers and projections, two of the above-mentioned buttons, or two flexible housing portions. The button can comprise an elastic material in order to automatically return to an original arrangement of the button after being pushed into the first housing.

In some embodiments, the ampule breaker can be connected to the cartridge in such a stable manner that the actuation of the breaking element does not impair the fluidic connection between the ampule breaker and the cartridge. It can thereby be ensured that a substantially complete transfer of a liquid from the ampule breaker into the cartridge can take place with the aid of the channel.

The device furthermore comprises a cartridge which is configured to receive a powder component of a bone cement. The cartridge comprises a second housing having a second interior. The cartridge can comprise a closure which closes the second interior. The closure can be connected to the second housing in a force-fitting or form-fitting manner. The closure can be releasably connected to the second housing. The closure can, for example, comprise a thread or a snap closure. As shown in more detail below, the closure can comprise a valve and/or a connection for a vacuum source.

The second housing can be designed in one or more parts. If the second housing is designed in several parts, a connection element can be arranged between two housing parts of the second housing, which connection element connects the two housing parts of the second housing to one another. The connecting element preferably releasably connects the two housing parts of the second housing to one another, for example in a force-fitting or form-fitting manner. The connecting element can, for example, comprise a thread or a snap closure.

The closure can have a feedthrough for a mixing rod.

The first housing and/or the second housing can be transparent or translucent. This can facilitate visual tracking of the movement of a liquid within the device.

The device can comprise a mixer that can be operated from the outside. The mixer can be arranged in and/or on the cartridge. The mixer can comprise a handle, a mixing rod, and a disc, wherein the pane can be arranged within the second housing. The mixer can be designed and configured to mix a liquid and a powder.

The cartridge can have a dispensing plunger or be connectible to a dispensing plunger. The dispensing plunger can be arranged movably in the second housing. The dispensing plunger can be designed and configured to dispense a bone cement from the cartridge, for example a PMMA bone cement.

The device comprises a channel which fluidically connects the first interior of the ampule breaker to the second interior of the cartridge. The channel preferably comprises an inlet which connects the first interior of the ampule breaker, in particular the collection region, to the channel. The channel preferably comprises an outlet which connects the channel to the second interior of the cartridge. The channel preferably comprises a central axis M, which is formed by the shortest connection of the center point of the inlet to the center point of the outlet and corresponds to the intended flow direction of a liquid through the channel. This central axis M can form an angle α with an outer wall of the cartridge. The angle α can preferably be 60° to 90°, for example 60° to 90°, 70° to 90°, 80° to 90°, 60° to 80°, 60° to 70°, or 70° to 80°. Accordingly, the fluid-conducting channel can preferably be arranged at an angle α of 60° to 90° relative to the second housing.

The channel can preferably have an inner diameter of less than 3 mm, preferably less than 2 mm and more preferably less than 1 mm, at least at one point. Due to a small inner diameter, the channel can be configured to form a meniscus by means of a liquid received in the channel. Such a meniscus can retain a liquid in the channel as long as no pressure difference prevails between the first interior and the second interior of the device. As a result, the dispensing of a liquid after the breaking open of a glass ampule in the ampule breaker can be better controlled in terms of time. This can have the advantage of preventing liquid from ingressing into the second interior too early, in order, for example, to prevent undesired adhesion to the channel by the bone cement.

In one embodiment, the device can comprise two ampule crushers, which can each be connected to the cartridge with a separate channel. In one embodiment, the two channels are arranged at an angle of 180° to one another. The device can have a mirror-symmetrical or point-symmetrical shape, for example with respect to the two ampule crushers and the two channels.

If the device comprises a closure, the channel can be arranged on the side of the second housing opposite the closure.

The ampule breaker preferably comprises a first end and a second end, wherein the first end and the second end are preferably arranged opposite one another. The second end is preferably configured to be oriented downward during the use of the device in order to dispense the liquid contained in a glass ampule downward into the channel within the first housing. In this context, “down” denotes the direction of gravity. “Up” in a corresponding manner denotes the opposite direction to gravity. Accordingly, the device can preferably be configured to be aligned with the first end upwards during use of the device.

The ampule breaker comprises a first housing in which the individual elements of the ampule breaker described herein can be arranged. The first housing comprises a first interior in which a holder for receiving a glass ampule is arranged.

The ampule breaker is configured to receive a glass ampule. A glass ampule is a container made of glass, which can delimit a liquid contained therein to the outside in a gas-tight and liquid-tight manner. Glass ampules typically comprise a base body having a substantially cylindrical shape. The glass ampules described herein preferably comprise an ampule head which is connected by an ampule neck to the remaining glass body, i.e., the main body, of the glass ampule. The ampule neck has a smaller diameter than the main body. The ampule neck can comprise a predetermined breaking point, wherein the ampule head can be separated from the glass ampule by being broken off at said predetermined breaking point. The predetermined breaking point therefore defines an opening from which the liquid can flow out of the glass ampule after the ampule head has been broken off. The predetermined breaking point is preferably annular. The inner diameter of the predetermined breaking point can preferably be 4 to 7 mm, for example 5 to 6 mm. A diameter of the predetermined breaking point of more than 4 mm can facilitate the flow of the liquid located in the glass ampule. In this case, the liquid can flow completely out of the glass ampule even when the glass ampule is oriented vertically.

The outflow of the liquid can also be improved by the arrangement of the glass ampule in the holder. This improvement in the outflow of the liquid can comprise a higher flow rate or a more complete emptying of the glass ampule. Preferably, the holder can be configured to hold the glass ampule at an angle of 20° to 50° relative to the perpendicular (i.e., in the direction of gravity) during use of the device described herein. In one embodiment, the device is configured to break open and empty a glass ampule at an angle of 20° to 50° to the vertical.

In one embodiment, the device is configured to break open and empty a glass ampule arranged at an angle of 20° to 50° to a longitudinal axis of the cartridge.

An angled arrangement as described above also allows the liquid to exit the glass ampule in cases in which the inner diameter of the predetermined breaking point is less than 4 mm, for example less than 3 mm.

The use of glass ampules can be advantageous with respect to film bags, since glass ampules can completely prevent the monomer liquid from exiting the ampule by diffusion over a period of years and can be produced and filled cost-effectively.

The ampule breaker comprises a holder for receiving a glass ampule. The holder can facilitate a permanent mounting of a glass ampule in the device. The holder can, for example, be configured to fix a commercially available glass ampule in a force-fitting or form-fitting manner, so that the glass ampule can be positioned immovably in the holder. In one embodiment, after being received in the holder, one can no longer be moved within the first housing or can only be moved by means of a plunger provided for this purpose. For this purpose, the holder can comprise a locking mechanism for fixing a glass ampule. For example, the device for holding a glass ampule can be equipped with an elastic lug or strut which, for example, can be arranged in the direction of the first end of the ampule breaker.

In one embodiment, the holder is configured to receive a glass ampule at an angle of 20° to 50° to a longitudinal axis of the cartridge. In one embodiment, the holder is configured to receive a glass ampule at an angle of 0° to 20° to a longitudinal axis of the cartridge.

The device can furthermore comprise a collar which, for example, can be arranged in the direction of the second end of the ampule breaker. The collar can be dimensioned such that the ampule head can be pushed through the collar, wherein the collar restrains the base body of the ampule. A glass ampule can, for example, be held in the holder between such a collar and a lug or strut, or a comparable element for this purpose.

The device can further comprise a projection. The projection is preferably designed and configured to break open a glass ampule that can be received in the holder. For this purpose, the projection can be arranged in the first housing in such a way that it limits the free movement of a glass ampule that can be received in the holder. In particular, the projection can be arranged such that the head of a glass ampule that can be received in the holder collides with the projection when the glass ampule is moved within the device toward the second end of the ampule breaker. The projection can extend, for example, from the edge of the first housing toward the center of the interior of the first housing.

The device can furthermore comprise an outlet in order to dispense liquid from the channel of the first housing into the second housing. The outlet can comprise an opening, a nozzle, a tube or a hose line. Alternatively or additionally, the outlet can also comprise an adapter for connection to a cartridge with bone cement powder.

The ampule breaker can comprise a plunger movably arranged in the first housing. This plunger is preferably designed and configured to break open a glass ampule that can be received in the holder by pressure of the glass ampule against a projection. The plunger can move the holder in the direction of the projection in order to break off the head of the glass ampule from the base body of the glass ampule. Furthermore, the plunger is preferably designed and configured to pump a liquid from the glass ampule, which was broken open as described above, out of the outlet of the channel of the housing.

Accordingly, the plunger can carry out two functions, namely breaking opena glass ampule which can be received in the device, and dispensing the liquid contained in such a glass ampule from the ampule breaker to the cartridge.

The ampule breaker preferably further comprises a handle which allows a user to manually grip and move the plunger. The handle can, for example, be arranged at the first end of the ampule breaker, preferably at the end opposite the outlet of the ampule breaker.

In a preferred embodiment, the device comprises a valve. The valve can be configured to allow air to flow into the first housing or the second housing from the outside. Furthermore, the valve can be configured to pump a liquid from a glass ampule, which was broken open in the device, out of the housing by pressure of the plunger onto the air flowing into the housing. The valve can comprise a filter in order to prevent dust or germs from entering the device from the outside. The valve is preferably arranged on the ampule breaker, more preferably at the first end of the ampule breaker.

The valve can preferably be a one-way valve, i.e., a valve which is permeable to air in one direction and impermeable to air in another direction. The valve can be, for example, a ball valve, lip valve or plate valve.

The device can further comprise a catchment region. The catchment region can be arranged in the first housing and can be configured to receive a portion of a glass ampule that is broken off by the action of a breaking element. The catchment region is preferably dimensioned to be sufficiently large so that the ampule head of a glass ampule, which can be received in the device, can rotate laterally by approximately 90°, and the liquid contained therein can exit from the ampule head without completely breaking the ampule head itself. Examples of such a catchment region are described in EP2404864B1. The height and cross section of the catchment region are preferably at least as large as the height of the ampule head up to the predetermined breaking point, so that the catchment region is suitable for facilitating a rotation of the broken-off ampule head in the catchment region, as described in more detail in EP2404864B1.

In one embodiment, the device further comprises a screen which is arranged adjacently to the catchment region. The screen is preferably set up to retain fragments from a glass ampule broken open in the device, for example an ampule head, in the device. As a result, for example, the bone cement produced by means of the device can be prevented from being contaminated by glass fragments. The screen can be, for example, a metal wire mesh or a mesh made of a plastics material. The screen can also be a sheet made of metal or plastics material with expediently dimensioned holes. Furthermore, open-pore foams, such as Porex, can be used. Preferably, the screen should be made of a material which is not attacked by the liquid located in a glass ampule that can be received.

In a further embodiment, the device further comprises a collection region in order to receive liquid from a glass ampule broken open in the device. The collection region is preferably fluidically connected to the holder, so that liquid can flow from a glass ampule, which can be received in the holder, into the collection region. The liquid can preferably flow from the direction of the first end of the device toward the second end of the device. The collection region can be arranged and configured to receive a liquid from a glass ampule broken open in the device and to guide it to the outlet of the device. The collection region can be delimited from the catchment region by a screen as shown above, wherein the screen can form a fluidic connection between the catchment region and the collection region.

In one embodiment, the device is designed and configured for mixing bone cement. For this purpose, the device preferably further comprises a powder component for preparing a bone cement. This powder component can be arranged in a cartridge, wherein the cartridge can be fillable, via the channel, with liquid from a glass ampule broken open in the ampule breaker. An outlet of the channel can be connectible to the cartridge by means of an adapter, for example by a snap or screw connection. In order to mix the liquid and the powder component, a mixing element, for example a mixing rod, can further be provided which enables the liquid and the powder component to be mixed. The mixing rod can comprise a handle, a rod and a mixing disc.

In one embodiment, the ampule breaker comprises a first end and a second end, wherein the first end is arranged opposite the second end. A plunger is preferably arranged at the first end. This preferably relates to a portion of the plunger which forms a handle region for gripping and manually moving the plunger. This handle region is also referred to herein as “handle”. A channel is preferably arranged at the second end of the ampule breaker.

In one embodiment, the device comprises a plunger, and is designed and configured to pump the entire contents of a glass ampule, which can be received or has been received in the holder, out of the first housing into the second housing with a single stroke of the plunger. This can be achieved in particular by the fact that the stroke volume of the plunger is dimensioned to be sufficiently large in relation to the volume of a liquid to be dispensed in the glass ampule, for example at least the same size.

In one embodiment, a glass ampule is fastened or fixable in the holder in a force-fitting, form-fitting or integrally bonded manner. A glass ampule can, for example, be fixable or fastened in the device by adhesive bonding or clamping. As shown above, a glass ampule can be held by a collar, for example in the region of the ampule neck, and at the same time can be held under tension at the opposite floor of the ampule by a locking mechanism, for example a strut, a projection or the like.

In one embodiment, the ampule breaker is designed and configured to simultaneously break open and dispense liquid from two glass ampules. In one embodiment, the device is configured to simultaneously break open two glass ampules with the aid of a single plunger or with the aid of two connected plungers, and to dispense the liquid contained therein from the ampule breaker into the cartridge.

The device can further comprise an element for building a pressure difference between the first interior and the second interior in order to transfer a liquid through the channel. This element can be designed, for example, in the form of a plunger described herein. The device can also comprise a connection for connection to a pump. The device can comprise a vacuum connection for connection to a vacuum source that is typical in the medical environment. The device can comprise a mechanical or electric pump. The device can comprise a flexible element which can be configured to generate an overpressure in the first interior by pressing together the flexible element. This can take place, for example, in the manner of a Peleus ball which is usually used in laboratories as a pipetting aid. The element for building a pressure difference between the first interior and the second interior can be arranged in the ampule breaker or in the cartridge. The element can be operatively connected to the first interior or the second interior. An example of such an element for building a pressure difference is disclosed in WO 2010/089621 A1, which is hereby incorporated in full by reference. In one embodiment, the cartridge comprises a connection for a vacuum source. In one embodiment, the ampule breaker comprises a connection for a pressurized medium.

In one embodiment, the device comprises a glass ampule. The glass ampule can thereby be accommodated in the holder, as explained above. The glass ampule can contain a liquid, for example a monomer solution for producing a bone cement, preferably a PMMA bone cement.

If the device contains both a glass ampule with a liquid and a second component for producing a mixture thereof, for example a liquid and a powder for producing bone cement, or generally contains all starting materials for the intended purpose of the device, the device can also be referred to as a “full-prepacked mixing system”. It is accordingly immediately ready for use for producing bone cement without further starting materials having to be added. The device is used in particular for storing the two components and for mixing and dispensing bone cement, in particular polymethylmethacrylate bone cement. However, other fields of application in medical or non-medical fields are also conceivable in which a liquid is to be dispensed from a glass ampule. The powder can preferably be arranged in the cartridge, more preferably in the second housing, in particular in the second interior.

The device also comprises a cartridge. The cartridge is a closed container which is preferably configured for preparing, in particular for mixing, bone cement from a powder and a liquid. In one embodiment, the device comprises a connecting element in order to connect the ampule breaker to the cartridge. The connecting element can comprise a clamp in order to connect the first housing of the ampule breaker to the second housing of the cartridge. The device can also comprise an adapter for connecting the outlet of the ampule breaker to the cartridge. The cartridge can comprise a mixing rod for mixing a powder with a liquid which facilitates manual or machine mixing of the liquid and the powder component. The mixing rod can, for example, comprise a handle which can be arranged on an outer side of the device. The mixing rod can further comprise a rod which connects the handle to a sheet arranged within the second housing. The sheet can be moved within the second interior of the cartridge by a user of the mixing system with the aid of the handle in order to thereby mix the liquid with the powder component. The mixing rod can be wrested from the cartridge by means of a screw connection.

The cartridge comprises a housing which is referred to herein as a “second housing” in order to distinguish it from the housing of the ampule breaker, which is referred to herein as “first housing”. The second housing comprises a second interior. The second housing can optionally be of multipart design, as is illustrated in more detail in the drawings. The second housing can preferably have the shape of a hollow cylinder.

A further aspect relates to a method for dispensing a liquid from a glass ampule using a device as described above, wherein the method comprises the following steps,
fixing the glass ampule in a holder of the device;
breaking open the glass ampule using the breaking element;
dispensing the liquid from the broken-open glass ampule through a channel from a first interior in a first housing into a second interior of a second housing of the device,
wherein the fluid-conducting channel is arranged and configured to retain the liquid in a collection region, and to dispense the liquid to the second interior as soon as a pressure difference is applied between the collection region and the second interior.

In one embodiment, the method comprises a further step in which the liquid is mixed with a powder component in a second housing of the device. The method can be used, for example, to prepare a bone cement.

The liquid is preferably a monomer liquid for preparing a bone cement, for example a PMMA bone cement. The method is preferably a method for producing bone cement. The method is preferably carried out completely in vitro i.e., outside the human body.

The glass ampule can be broken open by means of a plunger which presses the glass ampule against a projection and thereby breaks off the ampule head. The glass ampule can also be broken open by means of a button which is pushed into the first housing, and thereby breaks off the ampule head. The glass ampule can also be broken open by means of a flexible part of the first housing, wherein the ampule head breaks off due to the bending of the first housing. Other methods known in the art for breaking open a glass ampule can also be used.

The device according to the invention is preferably a mixing system for bone cement, which comprises an ampule breaker described herein and a cartridge described herein. Such a mixing system can contain two components for producing bone cement, for example a monomer liquid and a powder component for producing PMMA bone cement.

An exemplary mixing system comprises, for example, an ampule breaker described herein, and also a mixing unit, comprising a hollow cylindrical cartridge having an interior, wherein a bone cement powder is arranged in the interior, and a cartridge head, which fluidically closes the interior at a proximal cartridge end, and a conduit means, which fluidically connects the interior of the mixing unit to the outlet of the device, wherein the conduit means preferably extends through a cartridge head feedthrough of the cartridge head of the mixing unit, and the conduit means and the cartridge head duct form a form-fitting connection.

In one embodiment, the mixing system comprises, for example,
an ampule breaker having a first housing, wherein the first housing comprises a first interior,
a holder arranged in the first housing for receiving a glass ampule,
a breaking element which is arranged in the first housing and is designed and configured to break open a glass ampule which can be received in the holder,
a collection region which is arranged in the first housing and is designed and configured to receive a liquid from a broken-open glass ampule,
a cartridge configured to receive a powder component of a bone cement, wherein the cartridge comprises a second housing having a second interior,
a fluid-conducting channel for dispensing a liquid from the collection region into the second interior, wherein the fluid-conducting channel is arranged and configured to retain the liquid in the collection region, and to dispense the liquid to the second interior as soon as a pressure difference is applied between the collection region and the second interior,
and a conduit means which fluidically connects the first interior of the mixing unit to the second interior, wherein the conduit means preferably extends through a feedthrough of the cartridge and the conduit means and the feedthrough form a form-fitting connection.

The feedthrough can be arranged on a cartridge head of the cartridge.

According to this embodiment, a glass ampule can be broken open by means of the breaking element, and the liquid contained therein can be transferred into the second interior by means of a conduit means.

The conduit means can, for example, be a hose connection which fluidically connects individual elements of the mixing system to one another within the mixing system. The conduit can preferably comprise or consist of an elastic polymer. The elastic polymer can comprise, for example, a silicone, PE, PP, PU, PA or FEP.

In a further embodiment, the mixing system comprises
an ampule breaker having a first housing, wherein the first housing comprises a first interior,
a holder arranged in the first housing for receiving a glass ampule,
a breaking element which is arranged in the first housing and is designed and configured to break open a glass ampule which can be received in the holder,
a collection region which is arranged in the first housing and is designed and configured to receive a liquid from a broken-open glass ampule,
a cartridge configured to receive a powder component of a bone cement, wherein the cartridge comprises a second housing having a second interior,
a fluid-conducting channel for dispensing a liquid from the collection region into the second interior, wherein the fluid-conducting channel is arranged and configured to retain the liquid in the collection region, and to dispense the liquid to the second interior as soon as a pressure difference is applied between the collection region and the second interior,
a cartridge head which fluidically closes the second interior at a proximal cartridge end,
and a hollow-cylindrical adapter which is configured to reversibly connect a proximal cartridge end to an outlet of the channel.

According to this embodiment, a glass ampule can be broken open by means of the breaking element, and the liquid contained therein can be transferred into the second interior by means of a reversibly connectible adapter.

In a further embodiment, the mixing system comprises
an ampule breaker having a first housing, wherein the first housing comprises a first interior,
a holder arranged in the first housing for receiving a glass ampule,
a breaking element which is arranged in the first housing and is designed and configured to break open a glass ampule which can be received in the holder,
a collection region which is arranged in the first housing and is designed and configured to receive a liquid from a broken-open glass ampule,
a cartridge configured to receive a powder component of a bone cement, wherein the cartridge comprises a second housing having a second interior,
a fluid-conducting channel for dispensing a liquid from the collection region into the second interior, wherein the fluid-conducting channel is arranged and configured to retain the liquid in the collection region, and to dispense the liquid to the second interior as soon as a pressure difference is applied between the collection region and the second interior,
a cartridge head which fluidically closes the second interior at a proximal cartridge end,
a foot section which is configured as a contact surface for the mixing system,
a cartridge head which is connectible or is connected to the foot section,
an injection nozzle which is arranged in the foot section and extends into the cartridge head,
and a conduit means which is fluidically connected to the injection nozzle.

According to this embodiment, a glass ampule can be broken open by means of the breaking element, and the liquid contained therein can be transferred via an injection nozzle into the second interior by means of a conduit means.

The mixing system is preferably a “full-prepacked mixing system” as described above. Such a mixing system can be provided in a sterile and hermetically sealed packaging. The mixing system itself and the contained bone cement components can also be provided in a sterile manner.

In one embodiment, by the movement of a plunger, a glass ampule received in the holder can be broken open by pressure against a projection in order to release a liquid contained therein, for example a monomer liquid for preparing bone cement, from the glass ampule. Furthermore, due to the movement of the plunger, this liquid can be pumped through the channel and optionally through a conduit means shown above, an adapter and/or an injection nozzle into the mixing unit, and mixed there with the bone cement powder arranged there in order to form a bone cement from the liquid and the powder component.

To prepare a bone cement, the liquid from a glass ampule broken open in the ampule breaker and the powder component can further be mixed together in the cartridge. For this purpose, the mixing system can comprise a mixing rod which enables manual or machine mixing of the liquid and of the powder component. The mixing rod can, for example, comprise a handle which can be arranged on the outside of the mixing system. The mixing rod can further comprise a rod which connects the handle to a sheet arranged within the mixing system. The sheet can be moved within the interior of the hollow cylindrical cartridge by a user of the mixing system with the aid of the handle in order to thereby mix the liquid with the powder component.

The mixing system may further comprise a screen to retain fragments of the broken-open ampule, as described in more detail above.

The mixing system can furthermore comprise a catchment region, as described above. The catchment region can be arranged in the housing and can be configured to receive a portion of a glass ampule that is broken off by the action of the plunger. The catchment region is preferably dimensioned to be sufficiently large so that the ampule head of a glass ampule, which can be received in the device, can rotate laterally by approximately 90°, and the liquid contained therein can exit from the ampule head without completely breaking the ampule head itself.

### EXAMPLES

The invention is further illustrated below using examples which are, however, not to be understood as limiting. It will be apparent to a person skilled in the art that other equivalent means may be used similarly in place of the features described here.

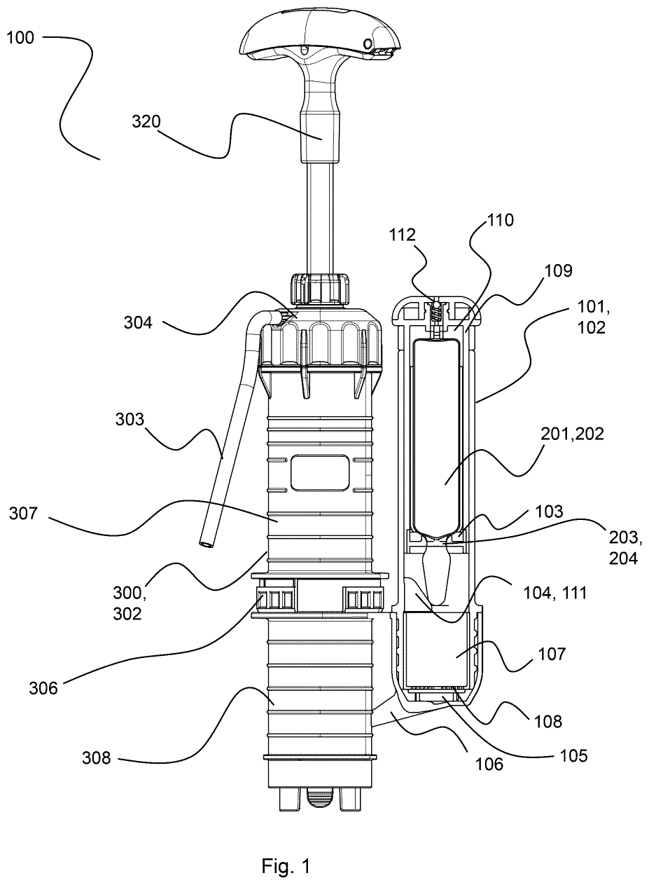

FIG. 1 shows by way of example a side view of a first embodiment of a device according to the invention 100, wherein the ampule breaker is shown in cross section. The device comprises an ampule breaker 101 having a first housing 102. The first housing 102 comprises a first interior 110 in which a holder 103 for receiving a glass ampule 201 is arranged. The glass ampule 201 comprises an ampule neck 203 having a predetermined breaking point 204. The glass ampule 201 contains a liquid 202. In the example shown here, the holder 103 comprises a collar which surrounds the glass ampule 201 in the region of the ampule neck 203. The ampule breaker 101 comprises a breaking element 104 which, in the example shown here, is designed as a projection 111. Here the ampule breaker 101 comprises a plunger 109 which is configured to push the glass ampule 201 against the projection 111 in order to break open the glass ampule 201. In the example shown here, the ampule breaker further comprises a valve 112 which can guide air from the outside into the first interior 110 but does not allow any air to reach the outside from the first interior 110. The plunger 109 can thus be used both for breaking open a glass ampule 201 and for pumping the liquid contained therein 202 out of the ampule breaker 101. The ampule breaker 101 comprises a catchment region 107 for receiving a broken-off ampule head. The catchment region 107 is limited downward by a screen 108 in order to retain the ampule head or other glass fragments of a broken-open glass ampule in the ampule breaker 101. The screen 108 is permeable to liquid 202. A collection region 105 is located on the other side of the screen 108 to receive the liquid 202. The screen 108 thus fluidically connects the catchment region 107 to the collection region 105. The collection region 105 is furthermore fluidically connected to a channel 106. The channel 106 fluidically connects the collection region 105 to a second interior 310 of a second housing 302 of a cartridge 300. The cartridge 300 comprises a second housing 302 which is designed in multiple parts in the example shown here. The second housing 302 thus comprises here a first portion 307 and a second portion 308, which are connected to one another by a connecting element 306, for example via a thread. The cartridge 300 is designed and configured to receive a powder, for example a powder component of a PMMA bone cement. The powder component can be arranged in the second interior 310. The cartridge 300 comprises a vacuum connection 303 in the form of a hose connector which facilitates the connection to a vacuum pump. In this way, a pressure difference between the first interior 110 and the second interior 310 can be applied in order to transfer the liquid 202 through the channel 106. The vacuum port 303 is arranged on a closure 304. The closure 304 closes the second housing 302 of the cartridge 300.

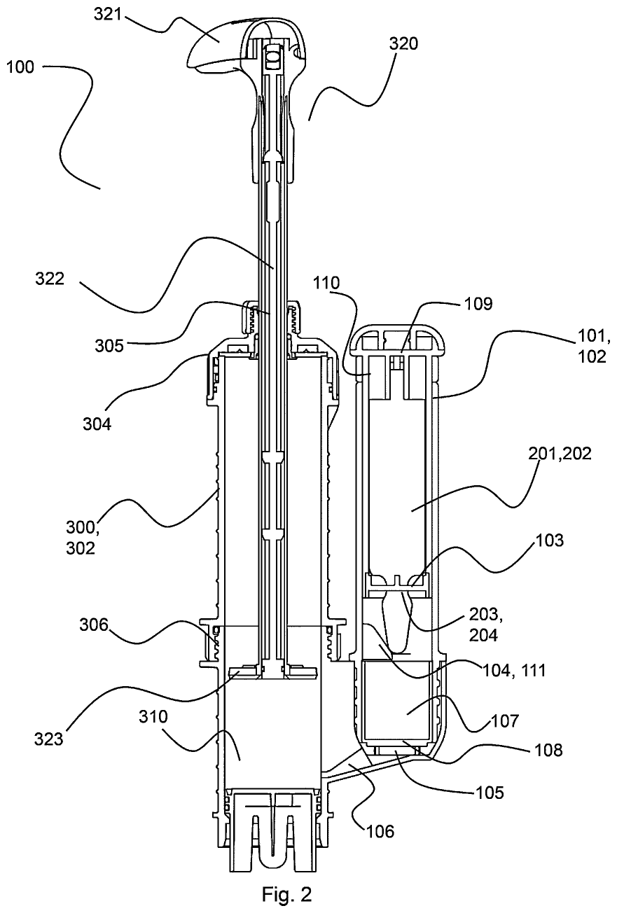

FIG. 2 shows a lateral cross-sectional view of an embodiment of a device 100 according to the invention. The device 100 comprises an ampule breaker 101, which is fluidically connected to a cartridge 300 via a channel 106. The cartridge 300 comprises a housing 302 having an interior 310. The housing 302 comprises a first portion 307 and a second portion 308, which are connected to one another via a connecting element 306, wherein the connecting element 306 comprises here a thread for connecting the first portion 307 and the second portion 308. The interior 310 is delimited by a closure 304. The closure 304 is screwed onto the housing 302 and thus releasably connected to the housing 302. The closure 304 also comprises a feedthrough 305 to receive a mixer 320. The mixer 320 comprises a handle 321, a rod 322 and a sheet 323, wherein the handle 321 is arranged outside the second housing 302 and the sheet 323 is arranged within the second housing 302. The mixer 320 facilitates the mixing of the liquid 202 with a powder component within the housing 302.

The ampule breaker 101 comprises a plunger 109 which can press a glass ampule 201 received in a holder 103 against a projection 111 which can be used here as breaking element 104. The travel path of the plunger 109 is limited by the projection 111. The plunger 109 thus serves to break open the glass ampule 201. In addition, using the plunger 109, a pressure difference can be generated between the first interior 110 and the second interior 310 in order to transfer a liquid 202 through a channel 106 into the cartridge 300.

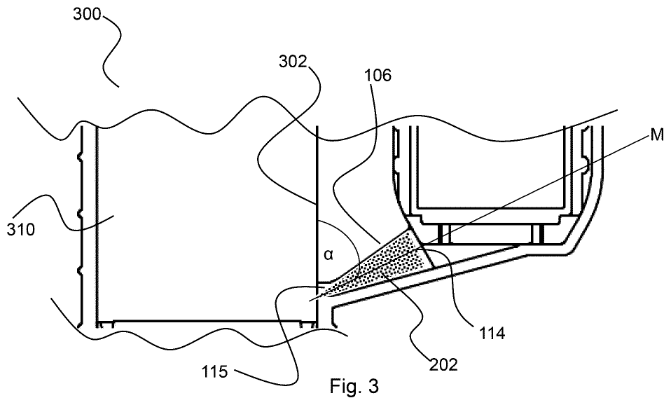

FIG. 3 shows a cross-sectional view of a detail of a device according to the invention. The channel 106 comprises an inlet 114 to receive a liquid from the ampule breaker. Furthermore, the channel comprises 106 an outlet 115 in order to dispense a liquid to the cartridge 300. Due to the small inner diameter of the channel 106, a meniscus is formed in the liquid 202 which keeps the liquid 202 in the channel 106. The formation of the meniscus is a function of the liquid used, the geometry of the channel, and its angle of inclination. An angle α is defined by the channel 106, more precisely by the central axis M of the channel 106, and the adjacent outer wall of the second housing 302. This angle is in a range of 60° to 90°. When conventional monomer solutions for PMMA bone cements are used, a meniscus is formed in the channel in this angle range, in particular when the channel has an inner diameter of 3 mm or less. Only by supplying the device with a pressure difference between the first interior 110 and the second interior 310 is the liquid 202 completely transferred through the channel 106 into the cartridge 300. The dispensing time of the liquid 202 into the cartridge 300, and thus the time at which the liquid makes contact with a powder component in the cartridge 300, can be controlled by the user.

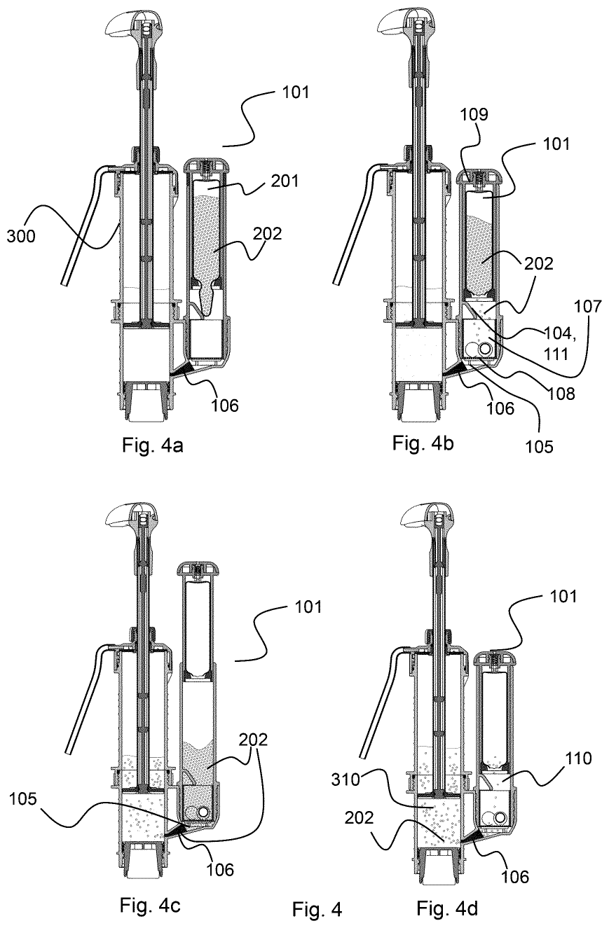

FIG. 4 shows a plurality of steps of a method in which a device according to the invention for producing a PMMA bone cement is used.

FIG. 4a shows a device according to the invention 100 in a first step of the method. A glass ampule 201 having a liquid contained therein 202, namely a monomer liquid for producing a PMMA bone cement, is received in an ampule breaker 101 of the device. The ampule breaker 101 is connected to a cartridge 300 via a channel 106 which contains a powder component for producing a PMMA bone cement.

FIG. 4b shows a device 100 according to the invention in a second step of the method. The glass ampule 201 is broken open with the aid of a breaking element 104 so that the liquid 202 contained can run out into a first interior 110. In the example shown here, this is done using a plunger 109 which presses the glass ampule 201 against a projection 111. The broken-off ampule head is retained in a catchment region while the liquid can reach a collection region 105 through a screen 108.

FIG. 4c shows a device according to the invention 100 in a third step of the method. The liquid 202 reaches the collection region 105 and the channel 106 fluidically connected thereto by gravitational flow and is retained by a meniscus of the liquid 202 being formed in the ampule breaker 101.

FIG. 4d shows a device according to the invention 100 in a fourth step of the method. By building a pressure difference between the first interior 110 and the second interior 310, liquid 202 can reach the second interior 310 in order to combine there to the powder component.

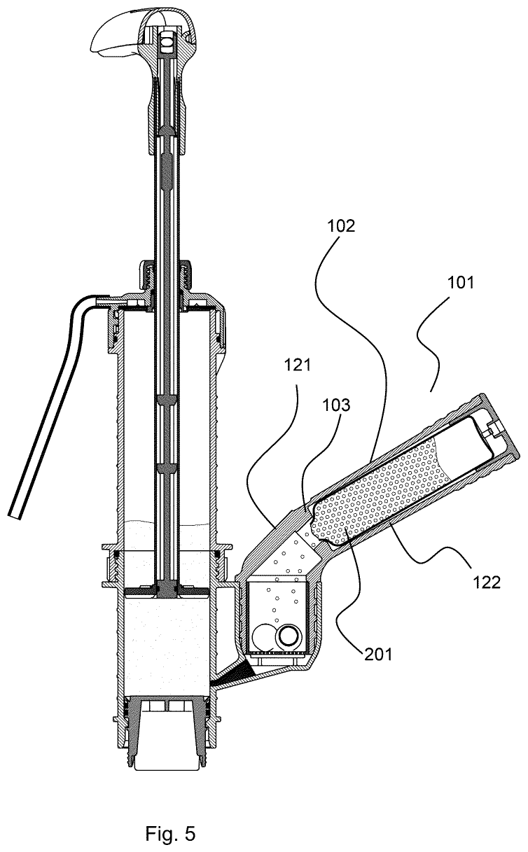

FIG. 5 shows a further embodiment of a device 100 according to the invention. In the embodiment shown here, the breaking element of the ampule breaker 101 is designed in the form of a flexible housing part 121 connected to a more rigid housing part 122. The flexible housing part 121 and the rigid housing part 122 are adjacent to one another in the first housing 102 of the ampule breaker 101 so that the neck 203 of a glass ampule 201 received in the holder 103 is located in the region of the transition between the flexible housing part 121 and the rigid housing part 122. By manually bending the flexible housing part 121 relative to the rigid housing part 122, an ampule 201 can be broken open.

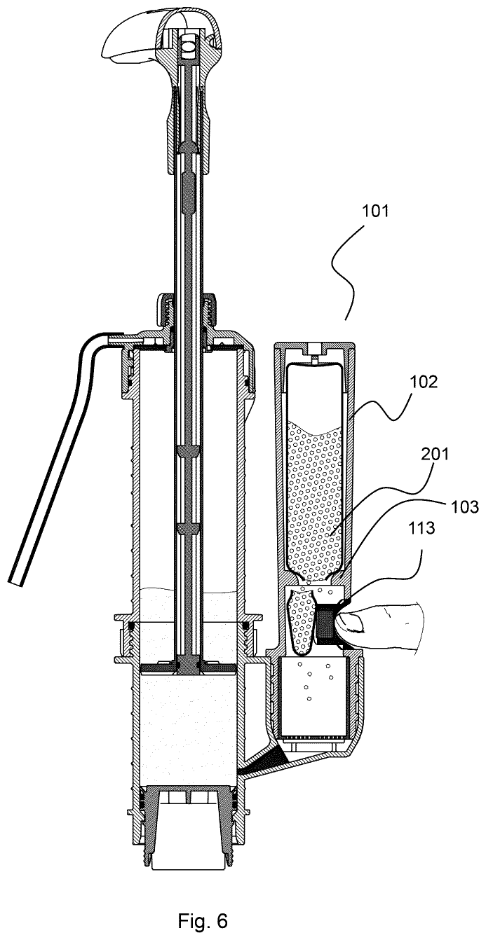

FIG. 6 shows a further embodiment of a device 100 according to the invention. In the embodiment shown here, the breaking element of the ampule breaker 101 is designed in the form of a button 113 which can be pushed into the first housing 102 from outside. The button 113 can be manually pushed against the head of a glass ampule 201 arranged in the holder 103 and the head is thereby broken off in order to break open the ampule 201. The button preferably comprises 113 an elastic element in order to return to its original shape and arrangement after being pushed into the housing 102.

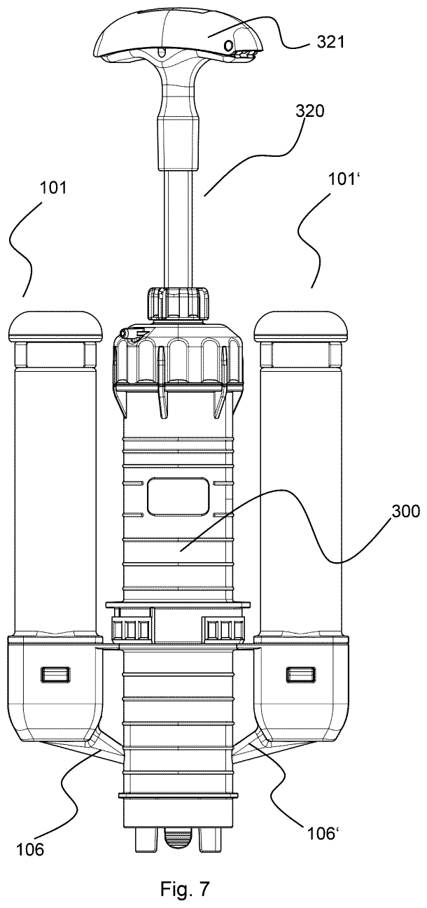

FIG. 7 shows a further embodiment of a device 100 according to the invention. In the embodiment shown here, the device comprises two ampule breakers 101,101′, which are each fluidically connected to the same cartridge 300 via a channel 106,106′. In such a device, for example, a total of two or a total of four glass ampules (i.e., two glass ampules per ampule breaker) can be broken open simultaneously and dispensed into the cartridge 300. One channel 106 is arranged inversely to the other channel 106′, wherein the two channels when viewed from above (i.e., from the direction of the handle 321 of the mixer 320) form a 180° angle with one another.

### LIST OF REFERENCE NUMERALS

100 device
101 ampule breaker
102 first housing
103 holder
104 breaking element
105 collection region (for liquid)
106 channel
107 catchment region (for glass breakage)
108 screen
109 plunger
110 first interior
111 projection
112 valve
113 button
114 inlet of the channel
115 outlet of the channel
121 flexible housing part
122 rigid housing part
201 glass ampule
202 liquid
203 ampule neck
204 predetermined breaking point
300 cartridge
302 second housing
303 vacuum connection
304 closure
305 feedthrough
306 connecting element
307 first portion of the second housing
308 second portion of the second housing
310 second interior
320 mixer
321 handle
322 rod
323 disc

1. A device for producing a bone cement, comprising:
an ampule breaker having a first housing, wherein the first housing comprises a first interior;
a holder arranged in the first housing for receiving a glass ampule;
a breaking element which is arranged in the first housing and is designed and configured to break open a glass ampule which can be received in the holder;
a collection region which is arranged in the first housing and is designed and configured to receive a liquid from a broken-open glass ampule;
a cartridge configured to receive a powder component of a bone cement, wherein the cartridge comprises a second housing having a second interior; and,
a fluid-conducting channel for dispensing a liquid from the collection region into the second interior, wherein the fluid-conducting channel is arranged and configured to retain the liquid in the collection region, and to dispense the liquid to the second interior as soon as a pressure difference is applied between the collection region and the second interior.

2. The device according to claim 1, further comprising a catchment region which is arranged in the first housing and is configured to receive a portion of a glass ampule that is broken off by the action of the breaking element.

3. The device according to claim 2, further comprising a screen which is arranged adjacent to the catchment region and is configured to retain fragments from a glass ampule broken open in the device in the ampule breaker.

4. The device according to claim 1, wherein the fluid-conducting channel is arranged at an angle α of 60° to 90° to the second housing.

5. The device according to claim 1, wherein the fluid-conducting channel has an inner diameter of less than 5 mm, preferably less than 4 mm or more preferably less than 3 mm, at least in portions.

6. The device according to claim 1, wherein a glass ampule is fastened or fixable in a force-fitting, form-fitting or integrally bonded manner in the holder, preferably fastened or fixable by adhesive bonding or clamping.

7. The device according to claim 6, wherein the glass ampule comprises an ampule neck having an annular predetermined breaking point, wherein the inner diameter of the predetermined breaking point is preferably 4 to 7 mm.

8. The device according to claim 1, wherein the cartridge further contains a powder component for preparing a bone cement, wherein the powder component is preferably arranged in the second interior of the second housing.

9. The device according to claim 1, wherein the ampule breaker is designed and configured for simultaneously breaking open and dispensing liquid from two glass ampules to the cartridge.

10. The device according to claim 1, wherein the breaking element comprises a plunger arranged movably in the first housing, and a projection, wherein the plunger is designed and configured to break open a glass ampule with a liquid, which glass ampule can be received in the holder, by pressure of the glass ampule against the projection, and to pump the liquid from the glass ampule through the channel out of the first housing into the second housing.

11. The device according to claim 10, wherein the ampule breaker further comprises a valve which is configured to allow air to flow into the first housing from the outside in order to subsequently pump a liquid from a glass ampule broken open in the device through the channel out of the first housing into the second housing by pressure of the plunger onto the air flowing into the first housing.

12. The device according to claim 11, wherein the valve is a one-way valve, preferably a ball valve, lip valve, or plate valve.

13. The device according to claim 10, wherein the ampule breaker is designed and configured to pump the entire contents of a glass ampule received in the holder through the channel out of the first housing into the second housing with a single stroke of the plunger.

14. The device according to claim 1, wherein the breaking element comprises a button which can be pushed into the first housing from the outside in order to break open a glass ampule, which can be received in the holder, by pushing the button into the first housing, wherein the button is preferably configured to automatically return to its original position after the button has been pushed in.

15. The device according to claim 1, wherein the first housing of the ampule breaker is at least partially flexible in order to break open a glass ampule that can be received in the holder by bending the first housing.
